(19) United States
(12) Patent Application Publication
(10) Pub. No.: US 2026/0174352 A1
(43) Pub. Date: Jun. 25, 2026

(54) METHOD AND SYSTEM FOR RESPIRATORY RATE ESTIMATION USING SINGLE-LEAD ELECTROCARDIOGRAMS

(71) Applicants: FONDATION BORDEAUX UNIVERSITÉ, BORDEAUX (FR); UNIVERSITÉ DE BORDEAUX, BORDEAUX (FR); THE GENERAL HOSPITAL CORPORATION, BOSTON, MA (US)

(72) Inventors: Kanchan KULKARNI, PESSAC (FR); Jesse D. ROBERTS, JR., MEDFORD, MA (US)

(21) Appl. No.: 18/990,342

(22) Filed: Dec. 20, 2024

(51) Int. Cl. A61B 5/08; A61B 5/00; A61B 5/28; A61B 5/352

(52) U.S. Cl. CPC A61B 5/0816; A61B 5/28; A61B 5/352; A61B 5/7264; A61B 2560/0443

(57) ABSTRACT

Computer-implemented method for estimating a respiratory rate of a subject, the method including acquiring a single-lead electrocardiographic signal from the subject, detecting R-peaks of the single-lead electrocardiographic signal, determining R-peak intervals from the R-peaks, extracting QRS complexes corresponding to the R-peaks based on the single-lead electrocardiographic signal, determining a time-domain sequence of root mean square amplitudes of the QRS complexes in a first window, the first window being in the time domain, generating, within a predetermined frequency range, a power spectrum of the sequence of root mean square amplitudes, determining a dominant frequency of the power spectrum, and estimating the respiratory rate of the subject based on the dominant frequency. Respirometry system employing same.

### BACKGROUND

Respiratory rate is a critical vital sign used to assess pulmonary function. Because of the importance of respiratory monitoring, methods for estimating respiratory rate using non-contact-based modalities and contact-based modalities have been explored.

Non-contact-based modalities such as contactless respiratory rate monitoring, which may entail radar, optical, or thermal sensors, might be useful for subjects whose contact-based monitoring is technically difficult, uncomfortable, or considered cumbersome and, hence, associated with poor compliance. However, devices employing these technologies are not broadly used. Contact-based modalities and methods of respiratory rate measurement are more clinically developed than non-contact methods. Contact-based methods estimate respiratory rate by measuring respiratory sounds, carbon dioxide levels in exhaled gases, respiratory gas flow, or changes in blood oxygen saturation (SpO2). In the latter case, pulse oximetry evaluates the SpO2 levels using photoplethysmography, quantifying changes in the optical absorption properties of hemoglobin with oscillations in blood flow. The pulsatile photoplethysmogram captured with these sensors has been used to estimate respiratory rate—but with limited accuracy—inductive plethysmography is the most accepted gold standard methodology used to estimate respiratory rates. However, this method involves the use of multiple sensor-bands, spread across the chest, to quantify rib cage and abdominal movement. Furthermore, the bands incorporate electrical wires, which need to be excited by low amplitude, high frequency alternating current. Additional electronic components are used to measure changes in the frequency of alternating current, indicative of the band movements during respiration, to estimate respiratory rate. This increases the complexity and expense of the equipment required to estimate respiratory rate using this method. These factors also limit the adaptability of this technology for remote monitoring where the bulky, specialized equipment would be cumbersome. Specialized technologies that use some of these non-contact- and contact-based technologies are already available in commercial devices that enable real-time respiratory rate monitoring. Yet, many of these devices depend on pulse oximetry or photoplethysmography to estimate respiratory rate, and reports suggest that the accuracy and precision of these devices for continuous respiratory rate monitoring have room for improvement.

Respiratory rate may be estimated based on electrocardiography. Electrocardiography-derived respiratory rate measurement is disclosed for example in the article “An optimized method for the estimation of the respiratory rate from electrocardiographic signals: implications for estimating minute ventilation” by Weiss et al., published in Am J Physiol Heart Circ Physiol, and in U.S. Pat. No. 11,771,330B2 and US2016/0331273A1—all of which rely on simultaneous acquisition of electrocardiographic signals via multiple electrocardiographic leads, thereby requiring relatively many electrodes deployed at multiple sites on the subject's body, and using such multi-lead signals to establish an estimate of the respiratory rate.

### SUMMARY

The inventors have recognized that respiratory rate estimating techniques relying on the ability to acquire multiple signals simultaneously via multiple electrocardiographic leads, and thus requiring relatively many electrodes deployed at multiple sites on the subject, require specialized and bulky instrumentation that may be cumbersome for subjects and unsuitable for mobile or remote health monitoring. In contrast, electrocardiography equipment suited for mobile and/or remote health monitoring may rely on fewer electrocardiographic leads—and thus fewer electrodes—to facilitate equipment portability. This reduced number of electrocardiographic leads renders them unsuitable for respiratory rate monitoring techniques that require multi-lead electrocardiographic signals.

To address this problem the inventors have developed a computer-implemented method for estimating a respiratory rate of a subject that is able to use a single-lead electrocardiographic signal. The method comprises acquiring a single-lead electrocardiographic signal from the subject, detecting R-peaks of the single-lead electrocardiographic signal, determining R-peak intervals from the R-peaks, extracting QRS complexes corresponding to the R-peaks based on the single-lead electrocardiographic signal, determining a time-domain sequence of root mean square amplitudes of the QRS complexes, generating a power spectrum of the sequence of root mean square amplitudes, determining a dominant frequency of the power spectrum, and estimating the respiratory rate of the subject based on the dominant frequency.

Thus, electrocardiographic estimation of respiratory rate may be possible even when multi-lead electrocardiographic signals are unavailable. The method may be deployed in equipment that is not capable of multi-lead electrocardiographic signal acquisition, or in equipment that is capable of multi-lead electrocardiographic signal acquisition. The method may allow equipment that is not capable of multi-lead electrocardiographic signal acquisition to be used nevertheless in estimation of respiratory rate. The method may allow equipment that is capable of multi-lead electrocardiographic signal acquisition to remain usable in the estimation of respiratory rate, even when a multi-lead signal is unavailable. Additionally, or alternatively, the method may allow equipment that is capable of multi-lead electrocardiographic signal acquisition to estimate respiratory rate based on a single-lead electrocardiographic signal, in parallel to multi-lead electrocardiographic signal-based estimation techniques.

The term “R-peak interval” may also be called an “R-R interval” and corresponds to a duration between two consecutive R-peaks within an electrocardiographic signal.

As used herein, the term “lead” denotes a comparison of electrical activities at two given points of the body of the subject. The electrical activity is measured using at least two electrodes.

As used herein, the term “electrode” denotes a conductor deployable to a subject, regardless of whether said conductor serves some purpose beyond measurement of electrical activity.

As used herein, “deployment” (and linguistic variants) of an electrode on a subject denotes electrical connection of the electrode to the subject to enable collection of an electrical signal from the subject via the electrode. Such collection may be done at an exterior surface delimiting the body of the subject and/or at an interior of the subject, that is to say inside the volume delimited by the exterior surface. Additionally, or alternatively, the connection may be performed through contact with between the electrode and the subject, by mounting the electrode transdermally on the subject (such as with via a port in the subject's body), and/or by implanting the electrode in the subject.

A given electrode is said herein to be “directly” used to measure an electrical activity at a given point when the position of the given point is the same as the position to which the given electrode is deployed. In contrast, an electrode is said herein to be “indirectly” used to measure an electrical activity when the electrical activity at that given point's position is determined using measurements of electrical activity obtained by multiple electrodes that are deployed at different positions from that given point's position.

As used herein, the term “single-lead electrocardiographic signal” denotes a single electrocardiographic signal corresponding to only one lead, that is to say a comparison between electrical activities, this comparison being measured between two given points of the subject's body. In contrast, the term “multi-lead electrocardiographic signal” denotes multiple electrocardiographic signals of cardiac electrical activity comprising a plurality of leads, a plurality of comparisons between electrical activities, each comparison being measured between two points of a given pair of points of the subject's body, the pairs of points being different. In both cases, the electrical activity at one of the points of a given lead may be measured directly or indirectly using an electrode, regardless of whether the electrical activity at the other of the points of the given lead is measured directly or indirectly.

The first window may be in the time domain.

The power spectrum may be generated for a predetermined frequency range.

The method may comprise deploying an electrocardiographic electrode on the subject prior to acquiring the single-lead electrocardiographic signal.

Optionally, the only electrode(s) deployed on the subject is/are the electrode(s) necessary for defining the lead of the single-lead electrocardiographic signal.

Limiting a total number of electrodes deployed on the subject may increase suitability for use with a mobile subject.

The respiratory rate may be estimated as being directly linearly related to the dominant frequency and inversely linearly related to a median value of the R-peak intervals of the single-lead electrocardiographic signal over an interval corresponding to the first window.

Changes in respiratory rate may be detectable based on changes in the dominant frequency and in the R-peak interval.

The respiratory rate may be estimated using the following formula:

$$R R = D F * 60000 / M R P I ;$$

in which RR is the respiratory rate, DF is the dominant frequency, and MRPI is a median value of the R-peaks intervals of the single-lead electrocardiographic signal over an interval corresponding to the first window.

The dominant frequency and the R-peak interval may be used to estimate the respiratory rate.

Estimating the respiratory rate of the subject based on the dominant frequency may comprise generating multiple preliminary respiratory rate estimations based on the dominant frequency, generating a secondary respiratory rate estimation by performing data smoothing based on a median value of the preliminary respiratory rate estimations over a moving second window spanning the multiple sequential preliminary respiratory rate estimations, and estimating the respiratory rate based on the secondary respiratory rate estimation.

Use of secondary respiratory rate estimations may enable data smoothing for example.

The second window may span sixteen preliminary respiratory rate estimations to generate the secondary respiratory rate estimations.

The duration of the second window may offer a tradeoff between an amount of context used when obtaining a given secondary respiratory rate estimation from a given preliminary respiratory rate estimation, and a responsiveness of secondary respiratory rate estimations to changes in the subject's respiratory rate.

Detecting the R-peaks of the single-lead electrocardiographic signal may comprise filtering the single-lead electrocardiographic signal to obtain a filtered electrocardiographic signal, R-peaks of the filtered electrocardiographic signal corresponding to R-peaks of the single-lead electrocardiographic signal, and detecting R-peaks present in the filtered electrocardiographic signal.

If noise is present in the acquired single-lead electrocardiographic signal, use of a filtered single-lead electrocardiographic signal may reduce problems arising from said noise.

Additionally or alternatively, detecting the R-peaks of the single-lead electrocardiographic signal may comprise applying, in the time domain, a third window to identify an individual heartbeat within the single-lead electrocardiographic signal, or within a filtered electrocardiographic signal corresponding to the single-lead electrocardiographic signal, the third window having a duration corresponding to an estimated duration of said individual heartbeat, applying, in the time domain, a fourth window, within the third window, to the single-lead electrocardiographic signal, or to the filtered electrocardiographic signal, to identify the individual heartbeat's corresponding QRS complex, identifying instances in the single-lead electrocardiographic signal, or in the filtered electrocardiographic signal, as the R-peaks where a magnitude corresponding to the fourth window exceeds a magnitude corresponding to the third window, the magnitude corresponding to the third window being representative of a moving average of the electrocardiographic signal calculated within the third window, the magnitude corresponding to the fourth window being representative of a moving average of the electrocardiographic signal calculated within the fourth window.

A given R-peak may be distinguished from possible random noise in the signal by consideration of trends within the signal rather than purely relying on instantaneous values of the signal.

Determining the time-domain sequence of root mean square amplitudes of the QRS complexes may include determining a root mean square amplitude for each of a plurality of extracted QRS complexes.

Each root mean square may be calculated within a fifth window centered on the corresponding QRS complex's R-peak.

The duration of the fifth window may be selected to exclude other portions of the signal, such as a P wave, a PR segment, a ST segment, a T wave, and/or a U wave.

The fifth window's duration may be eighty milliseconds.

Eighty milliseconds may correspond to a typical duration for a QRS complex (QRS interval) in an adult human subject.

The predetermined frequency range may be settable based on input from a user.

The user may be able to adjust the frequency range under consideration.

The predetermined frequency range may be greater than or equal to 1.8 breaths per minute and less than or equal to 80 breaths per minute, or is greater than or equal to 0.03 hertz and less than or equal to 1.34 hertz.

The predetermined frequency range may correspond to a typical range of respiratory rates observable in newborn, infant, children, and/or adult human subjects.

The predetermined frequency range may be less than or equal to 40 breaths per minute, or less than or equal to 0.67 hertz.

The predetermined frequency range may be less than or equal to 18 breaths per minute, or less than or equal to 0.3 hertz.

The predetermined frequency range may be greater than or equal to 5 breaths per minute, or greater than or equal to 0.08 hertz.

The first window may have a duration corresponding to a predetermined number of root mean square amplitudes in the sequence of root mean square amplitudes, and/or of a corresponding predetermined number of heartbeats in the single-lead electrocardiographic signal.

The duration may allow the first window to encompass sufficient data for estimating the subject's respiratory rate with a desired accuracy.

The inventors have also developed a respirometry system. The respirometry system includes one or more electrocardiographic electrode(s), in sufficient quantity for defining an electrocardiographic lead for monitoring a subject, and a processor configured to estimate a respiratory rate of the subject by executing a method as described above.

The system may be purpose-built for application of the method or may be retrofitted and/or reconfigured to enable application of the method.

The system may be portable by the subject.

Subject-portability may facilitate monitoring in a range of settings and/or improve subject comfort and/or compliance with monitoring.

The system may include a user interface.

The system may provide and/or accept information to/from a user relating to monitoring of the subject.

The user interface may be configured to allow a user to input the predetermined frequency range, a value for a length of the first window, a selection of an algorithm to implement for detection of R-peaks, one or more parameters employed in extraction of QRS complexes, a choice to activate or deactivate filtering of the single-lead electrocardiographic signal, and/or a choice to employ or forego use of smoothing in estimation of the respiratory rate based on the dominant frequency.

The user—which may be distinct from the subject, or which may also be the subject—may be able to adapt the method if needed.

The system may include a subject-side module, configured to be portable by the subject and to acquire the single-lead electrocardiographic signal from the subject, and a user-side module configured to execute at least a portion of the method in cooperation with the subject-side module.

The method may be implemented across multiple devices configured to cooperate with one another.

The user-side module may include the user interface such that the user is able to provide said input remotely from the subject-side module.

The method may be adaptable even remotely from the subject.

The system may be configured to indicate a margin of error for the estimated respiratory rate based on a calibration state of the system, and/or to determine a confidence interval for the estimated respiratory rate.

The system may be able to evaluate how reliable a respiratory rate estimation may be.

The system may be configured to monitor the subject using at most one electrocardiographic lead at any given time.

The system may be less cumbersome for the subject than a system that relies on multi-lead electrocardiography for estimation of respiratory rate.

The system may comprise electrocardiographic electrodes in sufficient quantity for defining multiple electrocardiographic leads for monitoring the subject. The system may be configured to acquire multiple single-lead electrocardiographic signals from the subject. The processor may be configured to estimate the respiratory rate of the subject based on any one of said single-lead electrocardiographic signals.

If the system is able to monitor the subject using multiple leads, the system may be able to acquire multiple single-lead electrocardiographic signals from said multiple leads and combine them as a sequence of portions of said signals wherein each instance in the sequence is reflective of only one of said signals.

### BRIEF DESCRIPTION OF THE FIGURES

The disclosure may be more completely understood in consideration of the following detailed description of aspects of the disclosure in connection with the accompanying drawings, in which:

FIG. 1 shows an exemplary acquired electrocardiographic signal.

FIG. 2 shows a block diagram representative of an exemplary method of estimating a respiratory rate.

FIG. 3 shows an exemplary filtered electrocardiographic signal.

FIG. 4 shows identification of a R-peak.

FIG. 5A shows an exemplary electrocardiographic signal with R-peaks thereof highlighted and an exemplary R-peak interval shown.

FIG. 5B shows an exemplary QRS complex.

FIG. 6 shows an exemplary series of root mean square amplitudes (represented as points) corresponding to an equivalent number of heartbeats'QRS complexes and a sliding time-domain window.

FIG. 7 shows an exemplary power spectrum representative of variations in root mean square amplitudes of heartbeats'QRS complexes with a dominant frequency of the power spectrum being highlighted by a star.

FIG. 8A shows an exemplary respirometry system.

FIG. 8B shows potential sites for electrode deployment on the body of a human subject.

FIG. 9 shows an exemplary subject-portable respirometry system.

FIG. 10 shows an exemplary respirometry system with a subject-side module and a user-side module.

The term “exemplary” is used herein in the sense of “example” rather than “ideal.” While aspects of the disclosure are amenable to various modifications and alternative forms, specifics thereof have been shown by way of example in the drawings and will be described in detail. It should be understood, however, that the intention is not to limit aspects of the disclosure to the particular embodiment(s) described. On the contrary, the intention of this disclosure is to cover all modifications, equivalents, and alternatives falling within the scope of the disclosure.

### DETAILED DESCRIPTION

As used in this disclosure and the appended claims, the singular forms “a”, “an”, and “the” include plural referents unless the content clearly dictates otherwise. As used in this disclosure and the appended claims, the term “or” is generally employed in its sense including “and/or” unless the content clearly dictates otherwise.

The following detailed description should be read with reference to the drawings. The detailed description and the drawings, which are not necessarily to scale, depict illustrative aspects and are not intended to limit the scope of the disclosure. The illustrative aspects depicted are intended only as exemplary.

Although the terms “first,” “second,” etc. may be used herein to describe various elements, components, regions, layers, sections, and/or parameters, these elements, components, regions, layers, sections, and/or parameters should not be limited by these terms. These terms are only used to distinguish one element, component, region, layer, or section from another region, layer, or section. Thus, a first element, component, region, layer, or section discussed herein could be termed a second element, component, region, layer, or section without departing from the teachings of the present inventive subject matter.

The disclosed technology relates to an electrocardiography-based respiratory rate estimation method and/or system that quantifies changes in the root mean square amplitude of electrocardiogram waveforms centered on peaks in the QRS complexes. The disclosed technology detects oscillations in the root mean square of a moving window of individual QRS complexes within a signal obtained from a single electrocardiographic lead. Broadly, the proposed technology offers a means of respiratory rate estimation based on electrocardiography, as opposed to direct measurements of respiratory movement, or as opposed to respiratory inductive plethysmography for example, which requires relatively cumbersome sensor deployment on the subject, or as opposed to photoplethysmography, which may not be as accurate and precise. Use of a single-lead electrocardiographic signal may offer one or several advantages over electrocardiography-based techniques that rely exclusively on multi-lead electrocardiographic signals.

Reliance on the root mean square amplitude of the QRS complex may allow the technology to retain accuracy across a wider variety of subject morphologies and/or pathologies than reliance on an individual feature of the QRS complex.

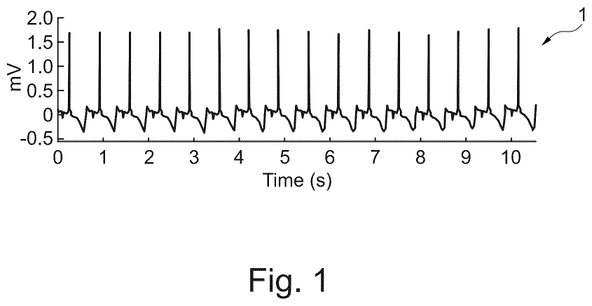

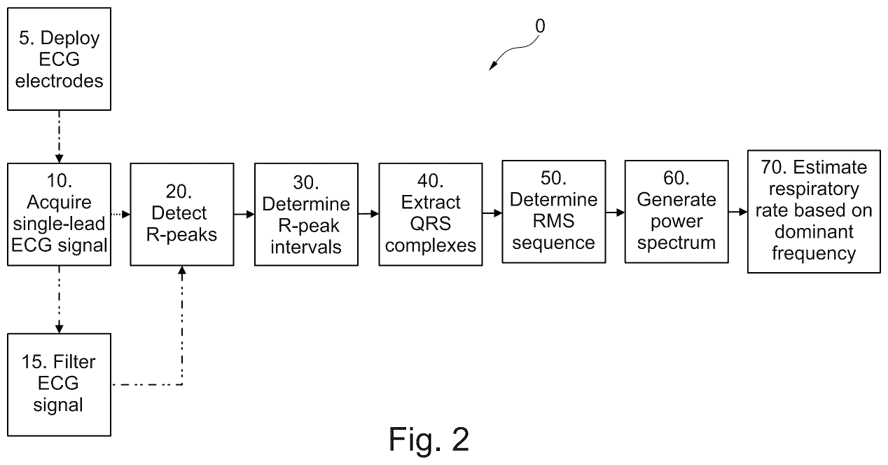

An exemplary single-lead electrocardiographic signal 1 is illustrated in FIG. 1. A block diagram of the method 0 is presented in FIG. 2, and is discussed with respect to the single-lead electrocardiographic signal 1 shown in FIG. 1.

Although the method 0 is executed primarily within the confines of a monitoring system, it is contemplated that the method includes a step of deploying 5 electrocardiographic electrodes on a subject to be monitored so as to define at least a single electrocardiographic lead. Such an option is represented in FIG. 2 using a dot-dash line (._._).

One of the electrodes may be deployed on the subject to measure electrical activity directly at one of the points of the lead. Alternatively, multiple electrodes may be deployed on the subject to measure electrical activity indirectly at one of the points of the lead - thereby defining a so-called “virtual electrode” positioned at the point. Non-limiting examples of virtual electrodes include Wilson's central terminal, Goldberger's central terminal, and/or any other position on and/or in the subject obtained based on the positions of where at least two electrodes are deployed on the subject. One or more electrodes are deployed on the subject to measure electrical activity—directly or indirectly—at the other of the points of the lead, regardless of whether electrical activity at the one of the points of the lead is measured directly or indirectly.

The single-lead electrocardiographic signal 1 is acquired 10 and subjected to processing 20, 30, 40, 50, 60, 70 to yield a respiratory rate estimate.

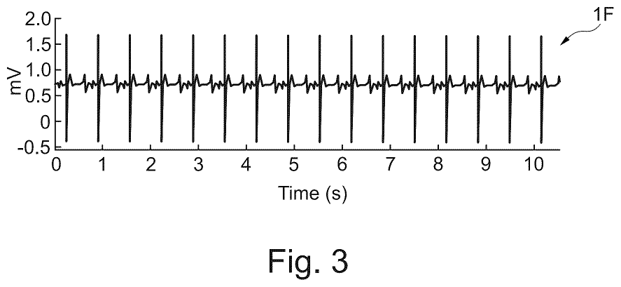

The acquired 10 single-lead electrocardiographic signal 1 may optionally be filtered 15 to eliminate noise and other artifacts. Such an option is represented in FIG. 2 using a dot-dot-dash line (.._.._). Such filtration 15 may yield a filtered single-lead electrocardiographic signal 1F, as illustrated in FIG. 3. Filtration 15 may for example be accomplished using an algorithmic band pass filter and/or one or more other methods for electrocardiographic signal processing. As a non-limiting example, the bandpass filter may have a band of 8 Hz or more to 40 Hz or less. Use of such a band may facilitate identification of QRS complexes within the single-lead electrocardiographic signal.

Returning now to FIG. 2, peaks associated with R-waves (also called “R-peaks”) are detected 20. Such detection 20 may be performed on the acquired electrocardiographic signal 1, or on the filtered signal 1F when applicable. The ability to perform such detection 20 using the acquired electrocardiographic signal 1 is represented using a dotted line (. . . . ), whereas the ability to perform such detection 20 using the filtered signal 1F is represented using the dot-dot-dash line discussed above.

For purposes of simplicity, as used herein, the reference numeral 101 denotes a signal that is equivalent to the single-lead electrocardiographic signal 1 obtained during acquisition 10 for instances when no filtering 15 is performed prior to the processing 20, 30, 40, 50, 60, 70 described hereafter, or to the filtered single-lead electrocardiographic signal 1F obtained via filtering 15 of the acquired 10 single-lead electrocardiographic signal 1 when filtering 15 is performed prior to the processing 20, 30, 40, 50, 60, 70 described hereafter.

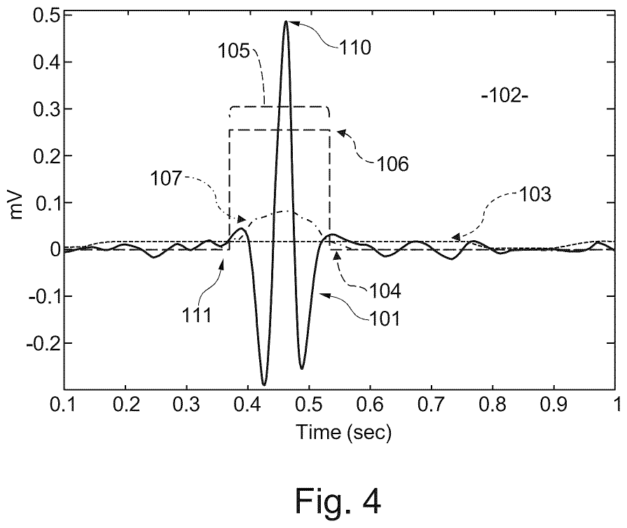

R-peak detection 20 may be accomplished using a two-averaging method, as illustrated in FIG. 4. The two-averaging method may be more robust than other R-peak detection 20 methods.

As a non-limiting example, the two-averaging R-peak detection method includes applying what may be referred to herein as a “heartbeat window” 102 to the signal 101 to identify an individual heartbeat within the signal 101, and applying what may be referred to herein as a “QRS window” 104 to identify that individual heartbeat's QRS complex 111.

The duration of the heartbeat window 102 may be selected based on an estimate of the subject's heartbeat duration, such that the duration of the heartbeat window 102 is long enough to encompass one entire heartbeat and short enough to avoid encompassing two QRS complexes 111.

The duration 105 of the QRS window 104 is shorter than that of the heartbeat window 102, and may be selected based on an estimate of the duration of the subject's QRS complex 110, such that the duration 105 of the QRS window 104 is long enough to encompass one entire QRS complex 111 and short enough to avoid encompassing more than one QRS complex 111.

The time-domain positions of the heartbeat window 102 and the QRS window 104 are variable and are applied over an interval sufficient to contain at least one heartbeat, or more generally over an interval sufficient to contain at least as many heartbeats as are necessary for estimating the subject's respiratory rate.

The R-peak 110 is presumed to occur within a portion of the signal 101 where a QRS window value 107 is greater than a heartbeat window value 103. The QRS window value 107 is a value reflective of a portion of the signal 101 contained in the QRS window 104. The heartbeat window value 103 is a value reflective of a portion of the signal 101 contained in the heartbeat window 102. As a non-limiting example, the QRS window value 107 may be a moving-average value of the signal 101 within the QRS window 104, and/or the heartbeat window value 103 may be a moving-average value of the signal 101 within the heartbeat window 102.

The R-peak 110 may occur at a local maximum of the signal 101 occurring over an interval where the QRS window value 107 exceeds the heartbeat window value 103.

Optionally, a threshold criterion 106 may be applied to exclude, for example, instances where the QRS window value 107 exceeds the heartbeat window value 103 over an interval that is too short to be reflective of a QRS complex 111.

However, it is contemplated that one or more other techniques for R-peak detection 20 may be used, as an alternative or complement to the two-averaging technique described herein. As a non-limiting example, such other technique(s) for R-peak detection 20 may be or include a Pan-Tompkins algorithm, a Hamilton algorithm, a Christov algorithm, and/or a stationary wavelet transform detector.

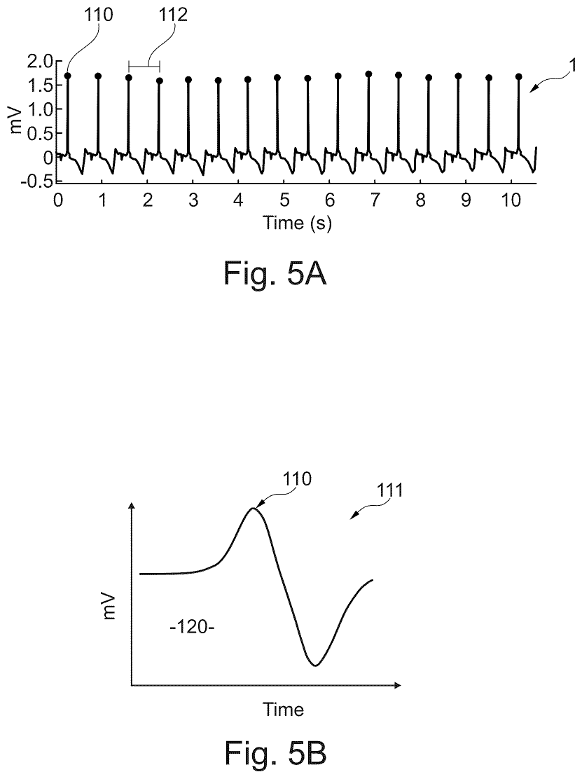

Returning now to FIG. 2, intervals between the R-peaks 110 in the signal 101 are determined 30. FIG. 5A illustrates the acquired single-lead electrocardiographic signal 1 shown in FIG. 1, wherein the R-peaks 110 are highlighted using dots and the R-peak interval 112 is represented as the duration between two successive R-peaks 110. In instances where the filtered signal 1F is used instead of the acquired signal 1, R-peaks 110 in the filtered signal 1F correspond to those of the acquired signal 1. In either case, the R-peak interval 112 corresponds to the duration between two successive R-peaks 110 of the signal 1, 1F, 101 in question.

Returning now to FIG. 2, QRS complexes of the signal 101 are extracted 40. As illustrated in FIG. 5B, a given QRS complex 111 may be extracted 40 by considering the signal 101 within what may be referred to as an “extraction window” 120 centered around the R-peak 110 of that given QRS complex 111. The extraction window 120 has a duration corresponding to the typical duration of a QRS complex 111. For example, the extraction window 120 may have a duration of 80 milliseconds (ms).

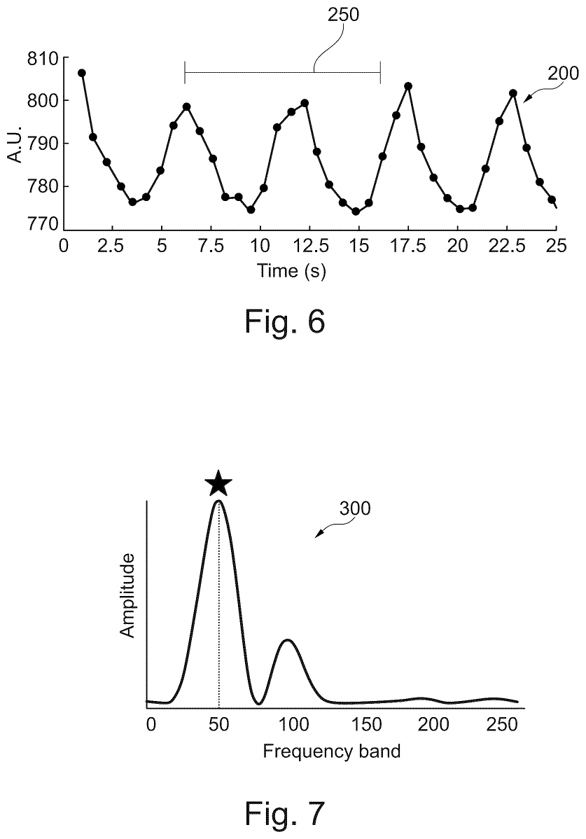

Returning now to FIG. 2, a root mean square amplitude of each extracted 40 QRS complex 111 in a series of heartbeats is calculated to determine 50 a sequence 200 of root mean square amplitude values, as illustrated in FIG. 6, whereby the Y-axis corresponds to the root mean square amplitude and the X-axis corresponds to the time when the R-peak 110 of the QRS complex 111 occurred. For ease of comprehension, the sequence 200 of root mean square amplitudes is represented in FIG. 6 as a rough curve, with the calculated amplitudes being represented as dots on the curve. Nevertheless, amplitudes can be seen to vary in an oscillatory manner over time.

Returning now to FIG. 2, a power spectrum 300 is generated 60 using the sequence 200 of root mean square amplitude values contained within what may be referred to as a “RMS window” 250. As illustrated in FIG. 6, the RMS window 250 can be understood as a time-domain window that is applied to the sequence 200 of root mean square amplitude values.

The duration of the RMS window 250 is determined to allow for the RMS window 250 to encompass a predetermined number of heartbeats'respective root mean square values sufficient for estimating the subject's respiratory rate. The duration may be determined based on a minimum anticipated heartrate of the subject. Optionally, the duration of the RMS window 250 may be selected to limit or avoid instances where the RMS window encompasses more heartbeats than are necessary for estimating the subject's respiratory rate.

The duration of the RMS window 250 may be determined based on a count of RMS values in the sequence 200 of root mean square amplitudes, and/or on a count of heartbeats in the signal 101, such that the RMS window 250's duration is reflective of a time-domain interval beginning with one heartbeat('s root mean square amplitude value's time coordinate) and ending with an Nth subsequent heartbeat('s root mean square value's time coordinate) where N+1 corresponds to the number of heartbeats(' root mean square amplitude values) necessary for estimating the subject's respiratory rate. Thus, the RMS window's duration may vary in the time domain as needed to encompass the predetermined number of root mean square amplitudes, regardless of the subject's underlying heartrate.

As non-limiting examples, the predetermined number of root mean square amplitudes may be:
greater than or equal to eight,
less than or equal to sixteen,
greater than sixteen,
less than or equal to thirty-two, and/or
less than or equal to sixty-four.

Lower predetermined numbers of root mean square amplitudes may tend to improve responsiveness to changes in respiratory rate at the expense of data smoothing; higher predetermined numbers of root mean square amplitudes may tend to improve smoothing of data at the expense of responsiveness. Estimation accuracy can be harmed if smoothing is excessive or insufficient. Use of sixteen as the predetermined number of root mean square amplitudes may offer an advantageous compromise between smoothness and responsiveness with neither insufficient nor excessive smoothing.

The time-domain position of the RMS window 250 can be fixed or variable. When using a variable time-domain position for the RMS window 250, the time-domain position is variable with a most-coarse granularity that is inferior to an anticipated heartbeat interval of the subject. Thus, as the RMS window moves from one position to another position in the time domain, previously non-encompassed root mean square amplitude values become encompassed in the RMS window 250 one at a time. When the time-domain position of the RMS window 250 is fixed instead of variable, estimation of the subject's respiratory rate at multiple instances in time can be achieved via the application of multiple RMS windows with encompassing different (overlapping or non-overlapping) time-domain intervals from one another.

FIG. 7 illustrates an exemplary power spectrum 300. The power spectrum is generated 60 over a frequency range corresponding to a range of anticipated respiratory rates for a given subject archetype and corresponds to the portion of the sequence 200 of root mean square amplitude values contained in the RMS window 250. As a non-limiting example, the frequency range may be 0.03 Hz or more (1.8 breaths per minute or more). Additionally, or alternatively, the frequency range may be 1.34 Hz or less (80 breaths per minute or less). Additionally or alternatively, the frequency range may be 0.67 Hz or less (40 breaths per minute or less). Additionally, or alternatively, the frequency range may be 0.08 Hz or more (5 breaths per minute or more). Additionally, or alternatively, the frequency range may be 0.3 Hz or less (18 breaths per minute or less).

As a non-limiting example, a subject archetype may correspond to a non-human subject, a human subject, a preterm subject, a partially-incubated subject, a neonatal subject, a hatchling subject, an infant subject, a juvenile subject, an adolescent subject, an adult subject, a geriatric subject, a conscious subject, a sedated subject, a comatose subject, an anesthetized subject, an exercising subject, a healthy subject, an obese subject, and/or an unhealthy subject.

The power spectrum 300 may be generated 60 using a Fourier transform, such as a discrete Fourier transform adapted for discrete signals such as the series of root mean square amplitudes, for example, a non-uniform discrete Fourier transform adapted for possible variations in intervals of time separating consecutive root mean square amplitudes. The power spectrum 300 presents a granularity in the frequency domain corresponding to a number of points employed in the associated Fourier transform. As a non-limiting example, the Fourier transform may employ 512 points, of which 256 points correspond to real frequencies represented in the power spectrum 300. Accordingly, although FIG. 7 shows 256 frequencies corresponding to a 512-point Fourier transform, other quantities of points and frequency bands are contemplated.

As seen in FIG. 7, the frequency range is represented within the power spectrum 300 as a predetermined number of frequency bands, wherein the lowest frequency band corresponds to the lower bound of the predetermined frequency range and the highest frequency band corresponds to the upper bound of the predetermined frequency range.

The inventors have determined that a relationship exists between the dominant frequency observable within a thusly generated 60 power spectrum 300 and the subject's respiratory rate. As seen in the example illustrated in FIG. 7, local maxima in the illustrated power spectrum 300 are observed around frequency band number 50, number 100, number 200, and number 250, with an absolute maximum (highlighted with a star) in the illustrated power spectrum 300 occurring around frequency band number 50. This absolute maximum corresponds to the dominant frequency within the predetermined frequency range.

Returning to FIG. 2, the dominant frequency obtained from the power spectrum 300 can be used to estimate 70 the respiratory rate. Such estimation 70 may be performed by means of the following equation:

$$R R = D F * 60000 / M R P I ;$$

wherein RR is the estimated respiratory rate, DF is the dominant frequency in the power spectrum 300, MRPI is a median value of the R-peaks intervals of the heartbeats corresponding to the root mean square amplitudes contained in the RMS window 250.

Although it is contemplated that the estimated respiratory rate RR obtained from the above equation be usable as a reliable estimation of the subject's respiratory rate, it is contemplated that the estimated respiratory rate RR obtained from the above equation be subject to further processing.

Accordingly, optionally, the estimated respiratory rate RR obtained from the above equation may be understood as a preliminary respiratory rate estimation, as opposed to a secondary respiratory rate estimation that would be obtained based on multiple such preliminary respiratory rate estimations.

As a non-limiting example, a given secondary respiratory rate estimation may be based on a series of preliminary respiratory rate estimations contained within what will be referred to herein as a “rate window.” Use of a series of preliminary respiratory rate estimations may enable processing such as data smoothing.

The series of preliminary respiratory rate estimations used for obtaining a given secondary respiratory rate estimation may contain a given preliminary respiratory rate estimation, corresponding to that secondary respiratory rate estimation in the time domain, and one or more preliminary respiratory rate estimations immediately preceding the given preliminary respiratory rate estimation, for example. Thus, each secondary respiratory rate estimation could consider a corresponding preliminary respiratory rate estimation within a context of one or more of the preliminary respiratory rate estimations that preceded it.

Additionally, or alternatively, the series of preliminary respiratory rate estimations may contain the given preliminary respiratory rate estimation and one or more preliminary respiratory rate estimations immediately following the given preliminary respiratory rate estimation, for example. Thus, if the preliminary respiratory rate estimations have been available with sufficient time prior to seeking the secondary respiratory rate estimation, it is possible to consider the corresponding preliminary rate estimation within a context reflective of one or more of the preliminary respiratory rates that succeeded it.

The series of preliminary respiratory rate estimations is contained within the rate window, which covers an interval that corresponds to a predetermined number of preliminary respiratory rate estimates and/or to an anticipated time-domain interval for capturing that predetermined number of preliminary respiratory rate estimates.

As a non-limiting example, the predetermined number of preliminary respiratory rate estimations in the series of preliminary respiratory rate estimates may be two, three, four, five, six, seven, eight, nine, ten, eleven, twelve, thirteen, fourteen, fifteen, sixteen, or more.

As a non-limiting example, a given secondary respiratory rate estimation may be based on a value representative of multiple preliminary respiratory rate estimations in the series. As a non-limiting example, the value representative of multiple preliminary respiratory rate estimations in the series may be or include moving average and/or a median value of said multiple preliminary respiratory rate estimations in the series. Use of a moving average of the preliminary respiratory rate estimations in the series may improve accuracy of a secondary respiratory rate estimation when the preliminary respiratory rate series is relatively uniform; use of a median value of the preliminary respiratory rate estimations in the series may facilitate exclusion of outliers and/or erroneous preliminary respiratory rate estimations.

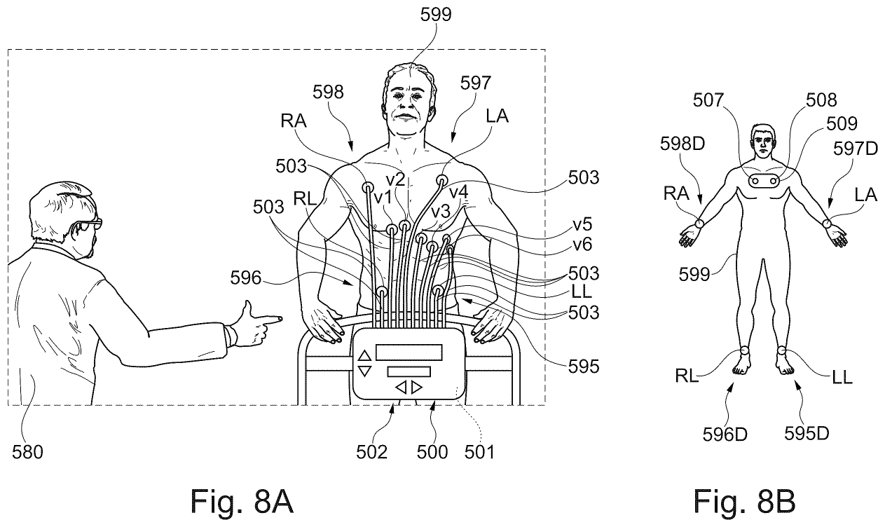

FIG. 8A shows an exemplary respirometry system 500 suitable for implementing the respiratory rate estimation method 0. The system 500 includes one or more electrocardiographic electrodes LA, LL, RA, RL, v1, v2, v3, v4, v5, v6 for acquiring 10 at least a single-lead electrocardiographic signal of the subject 599, and a processor 501 configured to estimate the respiratory rate of the subject 599 based on said signal by implementing the method 0.

The system 500 may for example comprise two electrodes LA, RA that are deployable on the subject 599 to define an electrocardiographic lead associated with the single-lead electrocardiographic signal acquired 10 via said electrodes LA, RA. As seen in FIG. 8A, the system 500 may comprise three, four, five, six, seven, eight, nine, or more electrocardiographic electrodes LA, LL, RA, RL, v1, v2, v3, v4, v5, v6, and may be capable of acquiring 10 multiple single-lead signals or even multi-lead signals. For example, the system 500 may include ten electrocardiographic leads usable for acquiring inter alia a twelve-lead electrocardiographic signal. For example, the system 500 may include electrodes in sufficient quantity for high-density body surface mapping. Regardless of quantity, the electrodes LA, LL, RA, RL, v1, v2, v3, v4, v5, v6 may be deployed 5 at locations on the subject 599 that are typically used for placement of electrodes for electrocardiography purposes.

The lead associated with the single-lead electrocardiographic signal 1, 1F, 101 may be defined between two electrodes LA, RA deployed on the body of the subject 599, or between a virtual electrode derived from multiple electrodes LA, RA, LL deployed on the body of the subject 599 and an additional electrode RL also deployed on the body of the subject 599.

When the system 500 comprises a sufficient number of electrodes LA, LL, RA, RL, v1, v2, v3, v4, v5, v6 for acquisition 10 of multiple single-lead electrocardiographic signals, and/or for acquisition of one or more multi-lead electrocardiographic signals in addition to acquisition 10 of one or more single-lead electrocardiographic signals, the system 500 may be configured to estimate the respiratory rate of the subject 599 using any individual electrocardiographic lead. Thus, if the system 500 is unable to obtain a multi-lead electrocardiographic signal, the system 500 may nevertheless be able to estimate respiratory rate using a single-lead electrocardiographic signal.

Additionally, or alternatively, if a single-lead electrocardiographic signal cannot be acquired 10 with respect to a preferred lead, the system 500 may be able to acquire 10 a single-lead electrocardiographic signal with respect to a non-preferred lead. As used herein, an indication that a given single-lead electrocardiographic signal is “associated with” (and variants) a given lead denotes is equivalent to an indication that said signal is “with respect to” (and variants) said lead.

The system 500 may be configured to implement the method 0 for estimating the respiratory rate of the subject 599 based on a time-domain sequence of single-lead electrocardiographic signals, such sequence being continuous over a time-domain interval sufficient for respiratory rate estimation, such sequence containing at most one single-lead electrocardiographic signal at any given time in said interval. Thus, if a given single-lead electrocardiographic signal is only intermittently available during that interval, the system 500 may still be able to obtain sufficient data to estimate the respiratory rate of the subject 599.

In FIG. 8A, the respirometry system 500 is illustrated as having ten electrodes LA, LL, RA, RL, v1, v2, v3, v4, v5, v6, deployable in a conventional manner on a subject 599 to define twelve electrocardiographic leads in a conventional manner, for example with one electrode RA corresponding to the right arm 598 of the subject 599, one electrode LA corresponding to the left arm 597 of the subject 599, one electrode RL corresponding to the right leg 596 of the subject 599, one electrode LL corresponding to the left leg 595 of the subject 599, and six precordial electrodes v1, v2, v3, v4, v5, v6 arranged on the chest of the subject 599 in a conventional manner.

Although in FIG. 8A, the electrode LA corresponding to the left arm 597, the electrode RA corresponding to the right arm 598, the electrode LL corresponding to the left leg 595, and the electrode RL corresponding to the right leg 596 are arranged proximally on their respective limbs 595, 596, 597, 598—or even on a portion of the torso of the subject 599 at an interface with said limbs 595, 596, 597, 598, it is contemplated for one or more of such electrodes LL, RL, LA, RA to be deployed on a distal portion of the corresponding limb 595, 596, 597, 598 of the subject 599—for example on a left ankle/foot/toe 595D, a left wrist/hand/finger 597D, a right ankle/foot/toe 596D, and/or a right wrist/hand/finger 598D, as illustrated in FIG. 8B.

Returning now to FIG. 8A, the respirometry system 500 may comprise a user interface 502 configured to allow a user 580 to input one or more parameters for use in the respiratory rate estimation method 0. Although the “user” 580 may be understood to be a different person or entity than the “subject” 599, it is also contemplated that the user 580 and the subject 599 be one and the same.

The parameter(s) input-able via the user interface 502 may be or may include one or more of:
the frequency range for which the power spectrum 300 is generated 60,
the duration of the heartbeat window 102 (for example, expressed as a time interval and/or as a number of heartbeats),
the duration of the QRS window 104,
the duration of the RMS window 250 (for example, expressed as a time interval and/or as a number of root mean square amplitude values and/or as a number of heartbeats),
the duration of the rate window (for example, expressed as a time interval and/or as a number of preliminary respiratory rate estimates),
a choice of whether the single-lead electrocardiographic signal 101 corresponds to the acquired signal 1 or to the filtered signal 1F—and optionally, what filtering 15 is applied to obtain the filtered signal 1F,
a choice of how or whether to generate secondary respiratory rate estimations,
an indication of the electrocardiographic lead employed for acquisition of the single-lead electrocardiographic signal (optionally expressed in terms of the electrodes LA, LL, RA, RL, v1, v2, v3, v4, v5, v6 used to define said lead).

The user interface 502 may additionally or alternatively be configured to display the estimated respiratory rate, a confidence interval, a margin of error, a readout of input from the user, information on system status, instructions for electrode deployment, and/or a combination thereof, for example.

FIG. 8A illustrates a respirometry system 500 suitable for use in a clinical setting, with signal wires 503 connecting the electrocardiographic electrodes LA, LL, RA, RL, v1, v2, v3, v4, v5, v6 to the processor 501 of the system 500. Such wires 503 allow for the transmission of signals from electrodes LA, LL, RA, RL, v1, v2, v3, v4, v5, v6 to the processor 501 and allow for the processor 501 to remain relatively stationary despite movement of the subject 599 and the electrodes LA, LL, RA, RL, v1, v2, v3, v4, v5, v6. However, such subject 599 movement may be relatively limited if the processor 501 is not subject-portable—for example, in FIG. 8A, monitoring is dependent on the subject 599 straying no further than a distance by which the signal wires 503 are able to connect the electrodes LA, LL, RA, RL, v1, v2, v3, v4, v5, v6 to the processor 501.

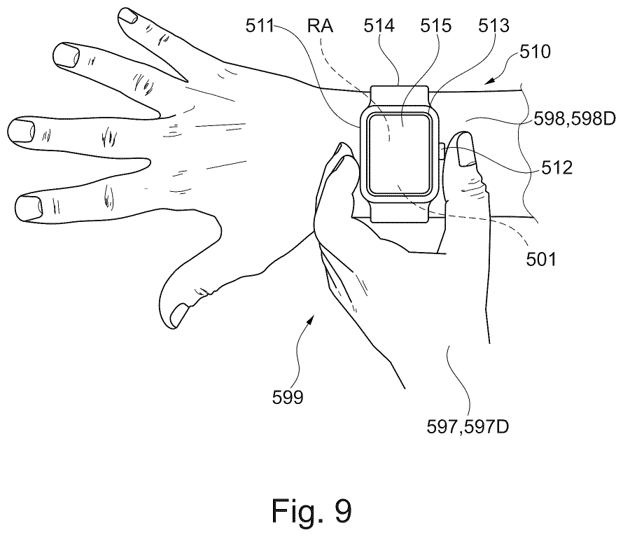

FIG. 9 shows an exemplary subject-portable respirometry system 510, which differs from the respirometry system 500 illustrated in FIG. 8A notably in that the processor 501 is subject-portable and in that system uses an ambulatory electrocardiography device to obtain the signal(s) upon which the respiratory rate estimation is based.

The ambulatory electrocardiography device may be or include a Holter monitor or any other ambulatory electrocardiography device.

To facilitate subject-portability, the system 510 may be configured to acquire 10 single-lead electrocardiographic signals rather than multi-lead electrocardiographic signals. Additionally, or alternatively, at least one of the electrodes RA via which the single-lead electrocardiographic signal is acquired 10 may be collocated with the processor 501. Additionally, or alternatively, the entire ambulatory electrocardiography device—including at least a second electrode LA via which the single-lead electrocardiographic signal is acquired 10—may be collocated with the processor 501.

The system 510 may be configured to be worn on the body of the subject 599 such that at least a first of the electrodes RA via which the single-lead electrocardiographic signal is acquired 10 is deployed 5 on the subject 599. Optionally, such first electrode RA is deployed 5 on a limb 597, 598 or extremity 597D, 598D thereof. Although FIG. 9 illustrates the subject-portable system 510 as being wearable as a wristwatch, the system 510 may be wearable as a ring, a glove/mitten, a bracelet, a bracer, an armband, a shoe, a sock, an anklet/ankle bracelet, a garter, or any other wearable item, such that the first electrode RA is deployed 5 on the finger, hand wrist, ankle, forearm, lower leg, upper arm, thigh, ankle, foot, toe, or other region(s) of the body of the subject 599 suitable for electrocardiographic electrode placement.

At least a second of the electrodes LA via which the single-lead electrocardiographic signal is acquired 10 is deployed 5—or arranged to be deployable—on the subject 599 during wear of the system 510 such that the second electrode LA and the first electrode RA define the lead associated with the acquired 10 single-lead electrocardiographic signal 1.

As a non-limiting example, the first and second electrodes LA, RA may be arranged such that the first electrode RA is deployed 5 to a first site on the body of the subject 599 during wear of the system 510, whereas the second electrode LA is arranged away from said first site on the body of the subject 599, at a position that is accessible by a second site on the body of the subject 599, which is generally opposite the heart of the subject 599 with respect to the first site. Deployment of the second electrode LA would then occur when the second site of the body of the subject 599 accesses the second electrode LA.

As a non-limiting example, the first electrode RA and second electrode LA may be arranged such that donning the system 510 causes the first electrode RA to contact the skin of the subject 599 at a first site and causes the second electrode LA to be remote from that first site, but contactable by a second site of the body of the subject 599 located laterally opposite from the first site so as to define an electrically-conductive path that traverses the heart of the subject 599.

As illustrated in FIG. 9, the first electrode RA may be arranged to contact the right arm 598 of the subject 599 when the system 510 is donned, and the second electrode LA may be arranged to be exposed when the system 510 is donned. To deploy 5 the second electrode LA, the left arm 598 is of the subject 599 brought into contact with the second electrode LA, thereby allowing the first and second electrodes LA, RA to define an electrocardiographic lead. If the first electrode RA has a positive polarity, and the second electrode LA has a negative polarity, the lead thus defined is typically referred to as “lead I”; otherwise, the lead thus defined returns a signal having inverted polarity with respect to a lead I single-lead electrocardiographic signal.

As used herein, a “lead I single-lead electrocardiographic signal” (and variants employing other lead names including but not limited to “lead II”, “lead III”, etc.) denotes a single-lead electrocardiographic signal associated with lead I (or the single-lead electrocardiographic signal associated with that named lead).

Similarly, the polarity of a signal associated with a given lead may be inverted by swapping the sites to which some or all electrodes that define said lead are deployed 5. Optionally, the system 510 may be configured to undo or otherwise accommodate signal inversion, automatically and/or based on input from the user 580.

Although the example illustrated in FIG. 9 is configured to estimate respiratory rate based on a lead I single-lead electrocardiographic signal, the system 510 may be configured to estimate respiratory rate based on a lead II single-lead electrocardiographic signal, a lead III single-lead electrocardiographic signal, and/or more generally any suitable single-lead electrocardiographic signal. The system 510 may be configured to employ a different parameter in the respiratory rate estimation method 0 when the estimation is based on a lead I single-lead electrocardiographic signal (for example) than on a single-lead electrocardiographic signal obtained from another lead. Such a parameter may be selectable via user 580 input, for example.

In the example illustrated in FIG. 9, the system 510 defines an opening in which a portion of the body of the subject 599 is receivable. When the body of the subject 599 is received in the opening, a so-called “inner surface” of the system 510, located on a wall of the opening, is arranged to face the skin of that portion of the body of the subject 599. The first electrode RA may be arranged on the inner surface of the system 510, such that donning the system 510 causes the first electrode RA to contact the skin of the portion of the body of the subject 599 received in the opening. The second electrode LA may be presented on a so-called “outer surface” of the system 510, so as to not be in contact with the portion of the body of the subject 599 that is received in the opening.

As a non-limiting example, the second electrode LA may be provided on an outer surface located in a portion of a frame 511, a crown 512, a bezel 513, and/or a strap 514 of the wristwatch/suitable wearable device.

When the subject-portable respirometry system 510 includes a user interface 502, such user interface 502 may also be collocated with the processor 501. For example, the user interface may be provided via a display 515 (and/or other component/s accessible to a wearer for interaction therewith) of the wristwatch/wearable device, optionally in a manner typical of so-called “smart watches”. Additionally, or alternatively, the user interface 502 may be provided remotely from the processor 501, for example, via a so-called “smartphone” or other suitable subject-portable computing terminal that is configured to communicate—wirelessly and/or via wires—with the processor 501.

The system 510 may optionally be implantable in the body of the subject 599. For example, the first electrode may be deployed to a first location in the thorax of the subject 599 or sub-dermally on the thorax of the subject 599, and the second electrode may be deployed to a second location in the thorax of the subject 599 or sub-dermally on the thorax of the subject 599, with the first and second locations being substantially on opposite sides (lateral, sagittal, transverse, etc.) of the heart of the subject 599 from one another so as to define an electrocardiographic lead. Optionally, a conductive (portion of) a casing of the system may serve as one of the electrodes of the system.

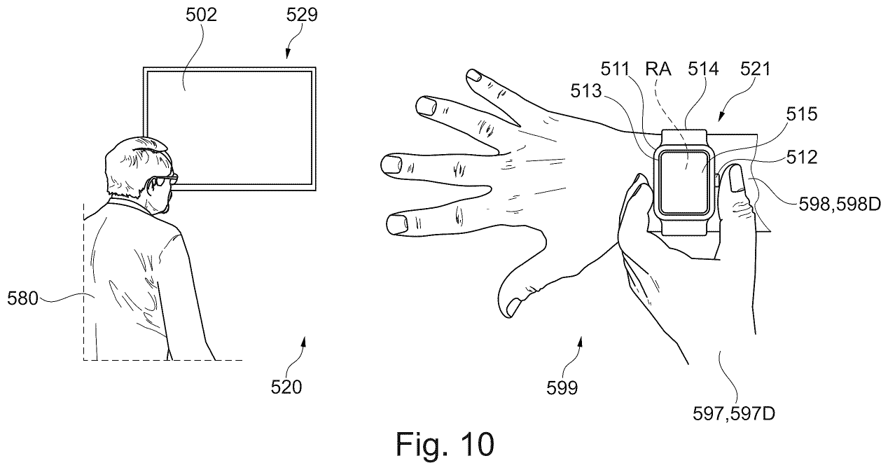

FIG. 10 illustrates an exemplary respirometry system 520 that includes a subject-side module 521 and a user-side module 529.

The subject-side module 521 is subject-portable and is configured to acquire at least one or more single-lead electrocardiographic signals in the same manner as described for the user-portable system 510 illustrated in FIG. 9 and/or described above.

The user-side module 529 is remote from the subject-side module 521 and is configured to perform at least a portion of the respiratory rate estimation method 0 disclosed herein. Said portion of the method 0 may be performed by the user-side module 529 substantially concurrently with signal acquisition 10 by the subject-side module 521, such that the subject's respiratory rate is estimated within five minutes, four minutes, three minutes, two minutes, one minute, forty-five seconds, thirty seconds, twenty seconds, fifteen seconds, ten seconds, five seconds, or less of the occurrence of the first R-peak 110 considered in the method 0. Additionally, or alternatively, said portion of the method 0 may be performed by the user-side module 529 substantially non-concurrently with signal acquisition 10 by the subject-side module, in which case the signal 1, 1F, 101 and/or one or more portion(s) thereof may be stored at least until said portion of the method 0 is performed by the user-side module.

The subject-side module 521 and the user-side module 529 are configured to communicate with each other wirelessly and/or via one or more wires. Such communication may occur directly between said modules 521, 529 and/or via one or more server(s), cloud(s), and/or other intermediary communication node(s).

The user-side module 529 may optionally include any user interface 502 described herein.

Optionally, the user-side module 529 may be configured to remain within a medical professional setting, such as a hospital, a clinic, a laboratory, or the like. For example, the user-side module 529 may be provided as a functionality of a computer system provided for such a medical professional setting. The user 580 may be able to adapt the method 0 remotely from the subject 599.

Optionally, the user-side module 529 may be subject-portable. For example, the user-side module may be provided as a functionality of a smartphone configured to cooperate with the subject-side module 521. Cooperation of the user-side module 529 and the subject-side module 521 to execute the respiratory rate estimation method 0 may enable compatibility of the method 0 with a wearable device that is suitable for at least acquiring 10 the single-lead electrocardiographic signal from the subject 599 but which might not be capable of executing the entire method 0 locally.

Electrodes may be arranged on the subject-side module 521 in the same manner as for the system 510 illustrated in FIG. 9 and/or disclosed above.

The subject-side module 521 may optionally be implantable in the body of the subject 599 in the same manner as for the system 510 illustrated in FIG. 9 and/or disclosed above. Optionally, a conductive (portion of) a casing of the subject-side module may serve as one of the electrodes of the system.

Optionally, the respirometry system 520 may include a subject-portable communication module configured to communicate with the user-side module 529 and the subject-side module 521 to facilitate cooperation between the subject-side module 521 and the user-side module 529. The communication module may be configured to communicate with the subject-side module using a first communication technique and to communicate with the user-side module using a second communication technique. The communication module may be provided whether the subject-side module is used on an exterior of the body of the subject 599 or is implanted in the body of the subject 599.

As mentioned above, it is contemplated to use any suitable single-lead electrocardiographic signal to estimate the respiratory rate. While the foregoing examples have largely discussed commonly-used electrocardiographic leads such as lead I, lead II, lead III, etc., such examples are not limiting—the single-lead electrocardiographic signal may be acquired for any suitable lead. As used herein, a lead is “suitable” when its points are sufficiently distant from one another—optionally relative to their respective distances from the heart of the subject—to allow for a difference in the electrical activity at their respective locations to be detected as a single-lead electrocardiographic signal, regardless of whether the respirometry system is entirely or at least partially subject-portable, includes a subject-portable module, is non-ambulatory, is non-subject-portable, etc., the examples disclosed herein may rely on any suitable single-lead electrocardiographic signal.

Accordingly, returning now to FIG. 8B, a different architecture 509 for a subject-portable respirometry system or subject-portable module of a respirometry system than those illustrated in FIGS. 9 and 10 can be seen, comprising at least two electrodes 507, 508 deployable on the subject 599 to define two points of a lead for the single-lead electrocardiographic signal. Such lead may or may not be different from the leads discussed in connection with FIGS. 8A, 9 and 10.

Whereas FIGS. 9 and 10 illustrate an architecture suited for deployment of electrodes to the subject's limb(s), such as a wristwatch for example, FIG. 8B illustrates a patch 509 that can be applied to the torso of the subject 599 to deploy the electrodes 507, 508, for example to skin over the ribcage of the subject.

Regardless of architecture, a given respirometry system may be configured to provide alerts should the estimated respiratory rate be below or above limits. Such limits may be user-settable.

Although the described embodiments were provided as different exemplary embodiments, it is envisioned that these embodiments are combinable or, when not conflicting, the features recited in the described embodiments may be interchangeable. Moreover, the features recited in the described embodiments are not inextricably linked to one another unless such a linkage is clearly indicated between two given features.

Throughout the description, including the claims, the term “comprising a” should be understood as being synonymous with “comprising at least one” unless otherwise stated. In addition, any range set forth herein, including the claims, should be understood as including its end value(s) unless otherwise stated. Specific values for described elements should be understood to be within accepted manufacturing or industry tolerances known to one of skill in the art, and any use of the terms “substantially” and/or “approximately” and/or “generally” should be understood to mean falling within such accepted tolerances.

Although the present disclosure herein has been described with reference to particular embodiments, it is to be understood that these embodiments are merely illustrative of the principles and applications of the present disclosure. The specification and examples are intended to be considered as exemplary only, with a true scope of the disclosure indicated by the following claims.

1. A computer-implemented method for estimating a respiratory rate of a subject, the method comprising:
acquiring a single-lead electrocardiographic signal from the subject,
detecting R-peaks of the single-lead electrocardiographic signal,
determining R-peak intervals from the R-peaks,
extracting QRS complexes corresponding to the R-peaks based on the single-lead electrocardiographic signal,
determining a time-domain sequence of root mean square amplitudes of the QRS complexes in a first window, the first window being in the time domain,
generating, within a predetermined frequency range, a power spectrum of the sequence of root mean square amplitudes,
determining a dominant frequency of the power spectrum, and
estimating the respiratory rate of the subject based on the dominant frequency.

2. The method of claim 1, comprising deploying electrocardiographic electrodes on the subject to define points of a lead prior to acquiring the single-lead electrocardiographic signal, said single-lead electrocardiographic signal being associated with said lead.

3. The method of claim 1, wherein only a quantity of electrocardiographic electrodes necessary to define points of a lead of the single-lead electrocardiographic signal are deployed on the subject.

4. The method of claim 1, wherein the respiratory rate is estimated as being directly linearly related to the dominant frequency and inversely linearly related to a median value of the R-peak intervals of the single-lead electrocardiographic signal over an interval corresponding to the first window.

5. The method of claim 4, wherein the respiratory rate is estimated using the following formula:
$$R R = D F * 60000 / M R P I ;$$
in which RR is the respiratory rate, DF is the dominant frequency, and MRPI is a median value of the R-peaks intervals of the single-lead electrocardiographic signal over an interval corresponding to the first window.

6. The method of claim 1, wherein estimating the respiratory rate of the subject based on the dominant frequency comprises:
generating multiple preliminary respiratory rate estimations based on the dominant frequency,
generating a secondary respiratory rate estimation by performing data smoothing based on a value representative of multiple sequential preliminary respiratory rate estimations over a moving second window spanning the multiple sequential preliminary respiratory rate estimations, and
estimating the respiratory rate based on the secondary respiratory rate estimation.

7. The method of claim 1, wherein detecting the R-peaks of the single-lead electrocardiographic signal comprises filtering the single-lead electrocardiographic signal to obtain a filtered electrocardiographic signal, R-peaks of the filtered electrocardiographic signal corresponding to R-peaks of the single-lead electrocardiographic signal, and detecting R-peaks present in the filtered electrocardiographic signal.

8. The method of claim 1, wherein detecting the R-peaks of the single-lead electrocardiographic signal comprises:
applying, in the time domain, a third window to identify an individual heartbeat within the single-lead electrocardiographic signal, or within a filtered electrocardiographic signal corresponding to the single-lead electrocardiographic signal, the third window having a duration corresponding to an estimated duration of said individual heartbeat,
applying, in the time domain, a fourth window, within the third window, to the single-lead electrocardiographic signal, or to the filtered electrocardiographic signal, to identify the individual heartbeat's corresponding QRS complex, and
identifying instances in the single-lead electrocardiographic signal, or in the filtered electrocardiographic signal, as the R-peaks where a magnitude corresponding to the fourth window exceeds a magnitude corresponding to the third window, the magnitude corresponding to the third window being representative of a moving average of the electrocardiographic signal calculated within the third window, the magnitude corresponding to the fourth window being representative of a moving average of the electrocardiographic signal calculated within the fourth window.

9. The method of claim 1, wherein determining the time-domain sequence of root mean square amplitudes of the QRS complexes includes determining a root mean square amplitude for each of a plurality of extracted QRS complexes.

10. The method of claim 1, wherein the predetermined frequency range is greater than or equal to 1.8 breaths per minute and less than or equal to 80 breaths per minute, or is greater than or equal to 0.03 hertz and less than or equal to 1.34 hertz.

11. The method of claim 10, wherein the predetermined frequency range is less than or equal to 18 breaths per minute, or less than or equal to 0.3 hertz.

12. The method of claim 10, wherein the predetermined frequency range is greater than or equal to 5 breaths per minute, or greater than or equal to 0.08 hertz.

13. The method of claim 1, wherein the first window has a duration corresponding to a predetermined number of root mean square amplitudes in the sequence of root mean square amplitudes.

14. A respirometry system comprising two electrocardiographic electrodes for monitoring a subject and a processor configured to estimate a respiratory rate of the subject by executing a method comprising:
acquiring a single-lead electrocardiographic signal from the subject by way of the electrocardiographic electrodes,
detecting R-peaks of the single-lead electrocardiographic signal,
determining R-peak intervals from the R-peaks,
extracting QRS complexes based on the single-lead electrocardiographic signal,
determining a time-domain sequence of root mean square amplitudes of the QRS complexes in a first window, the first window being in the time domain,
generating a power spectrum of the sequence of root mean square amplitudes,
determining a dominant frequency within a predetermined frequency range of the power spectrum, and
estimating the respiratory rate of the subject based on the dominant frequency.

15. The system of claim 14, wherein the system is configured to be portable by the subject.

16. The system of claim 14, comprising a user interface configured to allow a user to input:
the predetermined frequency range,
a value for a length of the first window,
a selection of an algorithm to implement for detection of R-peaks,
one or more parameters employed in extraction of QRS complexes,
a choice to activate or deactivate filtering of the single-lead electrocardiographic signal, and/or
a choice to employ or forego use of smoothing in estimation of the respiratory rate based on the dominant frequency.

17. The system of claim 16, comprising a subject-side module configured to be portable by the subject and a user-side module comprising the user interface such that the user may provide said input remotely from the subject-side module.

18. The system of claim 14, wherein the system is configured:
to indicate a margin of error for the estimated respiratory rate based on a calibration state of the system, and/or
to determine a confidence interval for the estimated respiratory rate.

19. The system of claim 14, said system being configured to monitor the subject using at most one electrocardiographic lead at any given time.

20. The system of claim 14, comprising multiple electrocardiographic electrodes for monitoring the subject, the system being configured to acquire multiple single-lead electrocardiographic signals from the subject, the processor being configured to estimate the respiratory rate of the subject based on any one of said single-lead electrocardiographic signals.
